(19) United States
(12) Patent Application Publication
(10) Pub. No.: US 2018/0153372 A1
(43) Pub. Date: Jun. 7, 2018

(54) DOORMAT WITH DECONTAMINATING DEVICE

(71) Applicant: Alessandra LATTERI, Catania (IT) (IT)

(72) Inventor: Alessandra LATTERI, Catania (IT) (IT)

(21) Appl. No.: 15/577,331

(22) PCT Filed: May 26, 2016
(86) PCT No.: PCT/IB2016/000718

§ 371 Date: Nov. 27, 2017

(30) Foreign Application Priority Data
May 26, 2015 (IT) 102015000018063

(51) Int. Cl. A47L 23/26; B08B 7/00; A61L 2/23

(52) U.S. Cl. CPC A47L 23/266; A61L 2202/17; A61L 2/23; B08B 7/0014

(57) ABSTRACT

A doormat with a decontaminating device comprises a base on which is applied a cleaning portion, including cleaning bristles or other like structure, and occupying a portion of the base, while leaving free a support zone adapted to receive a decontaminating device constituted by a block of sheet elements overlapping on one another, each sheet element being tearable away.

### BACKGROUND OF THE INVENTION

The present invention relates to a doormat including a device for decontaminating shoe soles.

As is known, shoe soles are easily contaminated by microorganisms of different genera, present on any surface, both in outdoor environments and in indoor environments.

The most common case of a contamination is obviously that occurring on a road, this contamination being easily transferred in both domestic and public residing environments.

Common doormats are designed for eliminating coarse dirt, but are not able to eliminate a bacterial load collected by a shoe outsole or sole.

Document WO 2005/084519 A1 substantially discloses the preamble of claim 1.

### SUMMARY OF THE INVENTION

The aim of the present invention is to provide a doormat including a decontaminating device adapted to eliminate or substantially reduce the contamination on shoe soles.

Within the scope of the above mentioned aim, a main object of the invention is to provide such a doormat which may be easily used by any user.

Another object of the present invention is to provide a versatile doormat which may be used both in domestic and public environments.

Yet another object of the present invention is to provide such a doormat which, owing to its specifically designed making characteristics, is very reliable and safe in operation.

The above mentioned aim and objects, as well as yet other objects, which will become more apparent hereinafter, are achieved by a doormat including a decontaminating device according to claim 1.

### BRIEF DESCRIPTION OF THE DRAWINGS

Further characteristics and advantages of the present invention will become more apparent hereinafter from the following disclosure of a preferred, though not exclusive, embodiment of the invention which is illustrated, by way of an indicative but not limitative example, in the accompanying drawings, where:

FIG. 1 is a perspective view of the doormat including the decontaminating device, according to the present invention;

FIG. 2 is a partially exploded perspective view of the doormat shown with the decontaminating device separated from the doormat base;

FIG. 3 is a further perspective view, similar to FIG. 2, showing a removal step for removing the last sheet arranged on a bottom portion of the decontaminating device;

FIG. 4 shows a step of applying the decontaminating device to the base support zone;

FIG. 5 shows a removal step of removing a top protective sheet;

FIG. 6 shows a use of the doormat for cleaning shoe outsoles or soles;

FIG. 7 shows a use of the subject doormat for decontaminating shoe soles on an active sheet of the decontaminating device;

FIG. 8 shows a removal step of removing the used sheet;

FIG. 9 shows a further removal step of removing the last sheet arranged at a bottom portion of a new decontaminating device to be applied to the base after having exhausted the first block;

FIG. 10 shows an application step of applying the new decontaminating device to the base support zone;

FIG. 11 shows a further removal step of removing the top protective sheet;

FIG. 12 shows yet another removal step of removing a cleaning portion from the base to wash the latter; and

FIG. 13 shows an applying step of applying the washed cleaning portion to the doormat base.

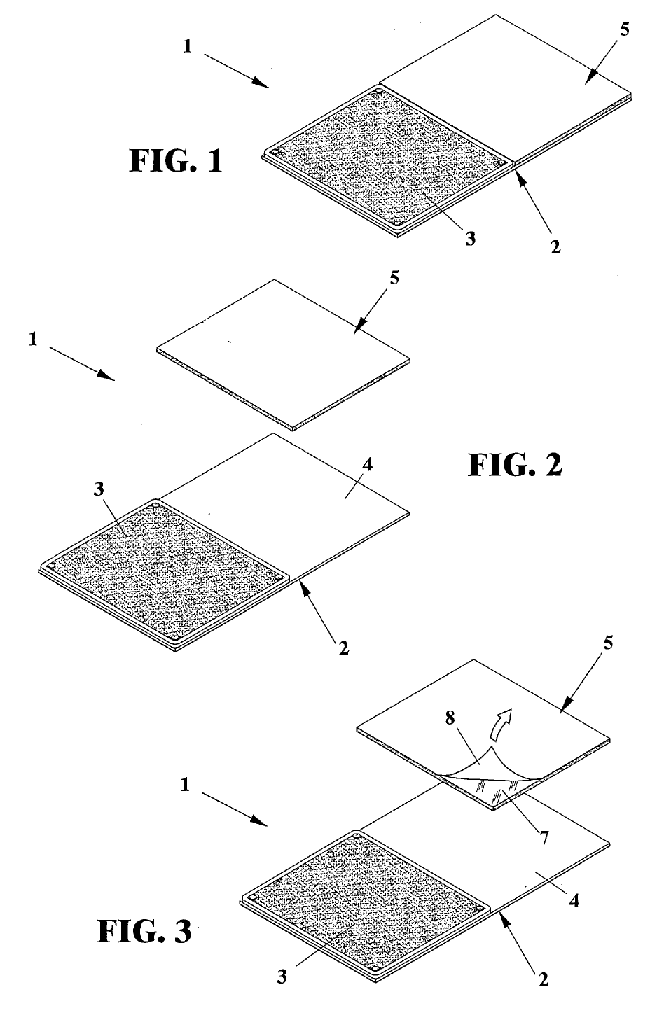

### DESCRIPTION OF THE PREFERRED EMBODIMENTS

With reference to the number references of the above mentioned figures, the doormat including a decontaminating device according to the present invention, which has been generally indicated by the reference number 1, comprises a base 2 on which a cleaning portion 3 is applied, said cleaning portion including a plurality of bristles or other like elements, occupying a portion of the base 2, while leaving free a support zone 4 adapted to receive a decontaminating device, generally indicated by the reference number 5.

The cleaning portion 3 is advantageously associated with the base 2 by means of snap fasteners 6, or other removable affixing systems as considered suitable.

The base 2 is preferably made of a rubber material, whereas the cleaning portion is advantageously made of a microfiber material, but it may also be made of other materials such as vegetable fibers or other natural or synthetic fibers.

The decontaminating device 5 comprises a block consisting of a plurality of sheet elements 7 overlapped onto one another.

The sheet elements 7 may be for example constituted by a polyethylene film impregnated by bacteriostatic and bactericidal agents.

The block of sheet elements 7 comprises a bottom film 8 and a top protective film 9.

Each said sheet element 7 comprises a sheet edge adapted to facilitate a tearing away and on which may be indicated the number of the sheet itself.

In fact, advantageously, it is provided to number the sheet elements thereby indicating to a user the number of the remaining bactericidal sheet elements which are still available.

The doormat may also comprise decorative elements of any desired type.

The use of the doormat according to the present invention is as follows.

The subject doormat may be arranged at an inlet of the environment to be maintained decontaminated, for example at a home doorway.

Before applying the decontaminating device 5 on the base 2, the last sheet element arranged at the bottom part of the sheet element block constituting the decontaminating device is removed, as is shown in FIG. 3.

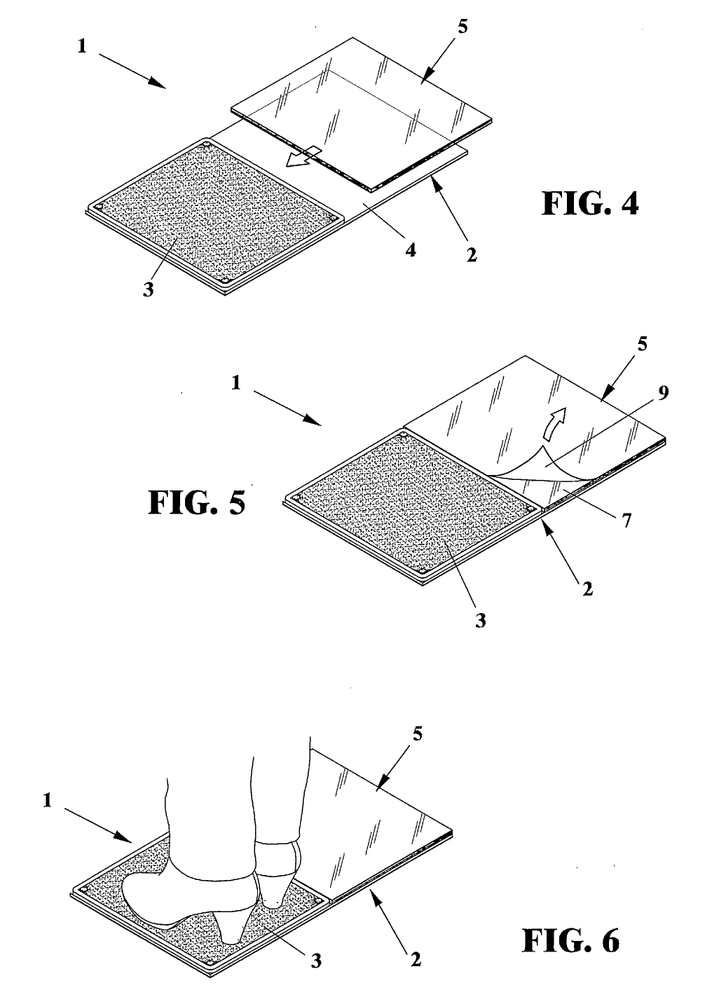

Then, the device 5 is applied to the base 2 at the support zone 4, by causing said device to completely adhere to the rubber base itself, as is shown in FIG. 4.

Before using the doormat, the protective top sheet element is removed, as shown in FIG. 5.

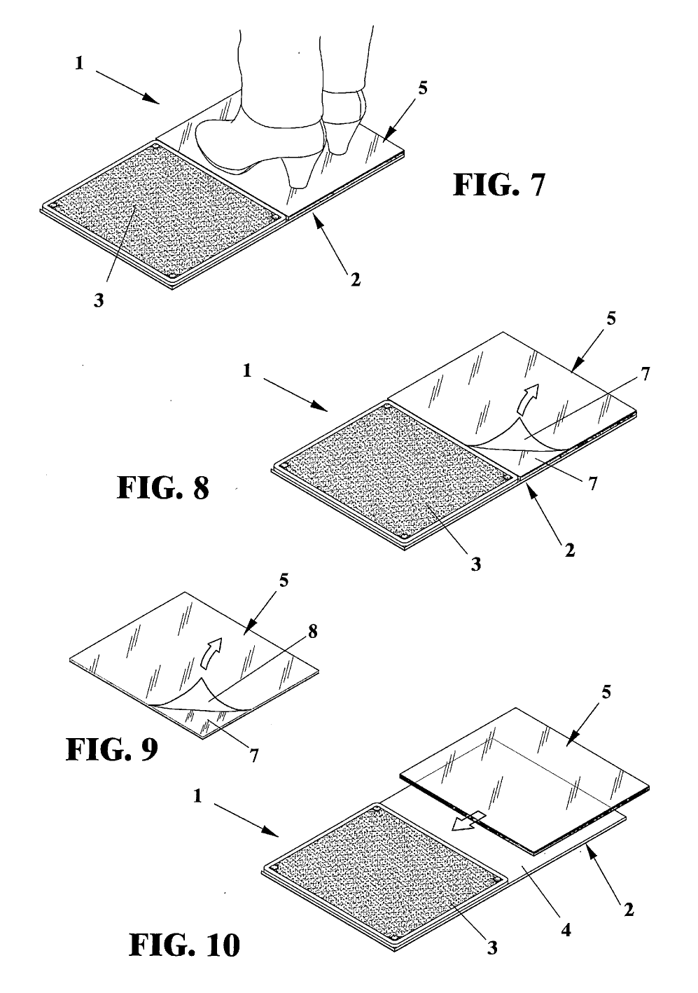

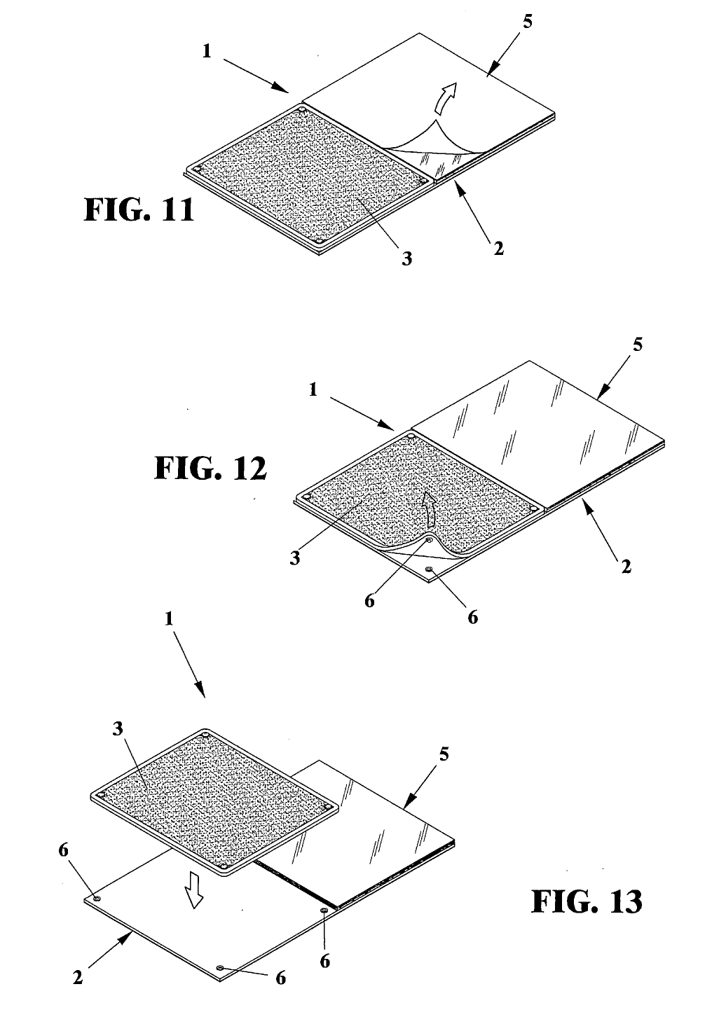

A proper use of the subject doormat provides that at first the shoe soles are cleaned on the cleaning portion 3 (FIG. 6) and then said soles being decontaminated by causing them to pass on the exposed sheet element of the device 5 (FIG. 7).

The decontaminating device 5 comprises a number of adhesive sheets, for example thirty adhesive sheets.

As the exposed sheet element has been exhausted, said sheet element is torn away and disposed of (FIG. 8), while leaving exposed the following sheet element which will accordingly be ready to provide the same decontaminating function.

A suitable numbered tongue is provided for facilitating the removal of the used sheet element while allowing to check the number of the decontaminating sheet elements left.

After having used all the adhesive sheet elements, the last sheet element is torn away from the support base 2, and a new device 5 is then applied, after removing the last sheet element arranged at the bottom part of the sheet block constituting the device itself, as is shown in FIG. 9.

Then, the new device 5 is applied to the base 2 at the support zone 4, by causing said device to fully adhere to the rubber base itself, as shown in FIG. 10.

Before using the subject doormat, the protective top sheet element is removed, as shown in FIG. 11.

It should be apparent that it is possible to wash the cleaning portion 3 every time it is desired.

To that end, as shown in FIG. 13, the cleaning portion 3 is detached from the base 2, owing to the snap fastener elements 6, and is put into a washing machine. After having been washed, the cleaning portion 3 may be easily and quickly relocated on the base 2, by locking it by means of the snap fastener elements 6.

It should be apparent that it is possible to replace the cleaning portion 3 with a new one when said cleaning portion is worn out.

In a cheaper variation, the doormat according to the present invention may be constituted by an “inlet” bristled doormat portion without the removable cleaning portion 3, that is the bristled portion of the doormat would constitute the starting portion of the doormat on which a user may carry out a starting cleaning of his/her shoe soles by said bristles, whereas the antibacterial sheet block may be a single block of films covering all the remaining width, that is the outlet width of the doormat, or may comprise at least two blocks of partial sheet elements, each being provided for decontaminating the sole of each shoe of the user.

It has been found that the invention fully achieves the intended aim and objects.

In fact, the invention has provided a doormat which may be used as a conventional doormat, but including an efficient decontaminating device providing an optimal antibacterial action.

The doormat including the decontaminating device according to the present invention allows to decontaminate from germs and bacteria any kind of environment, by providing optimal cleaning and hygienic characteristics.

In practicing the invention, the materials used, as well as the contingent size and shapes, can be any, depending on requirements.

1. A doormat with a decontaminating device (1) comprising a base (2) with a cleaning portion (3) provided with bristles or other like cleaning structure, occupying a portion of the base (2) while leaving free a support zone (4) adapted to receive a decontaminating device (5) constituted by a block of sheet elements (7) overlapped onto one another; each sheet element (7) being tearable away, characterized in that each said sheet element (7) comprises a tongue portion adapted to facilitate the tearing away of said sheet element and on which is indicated a progressive number adapted to indicate to a user the number of the bactericidal sheet elements (7) which are still available.

2. A doormat, according to claim 1, characterized in that each said sheet element (7) is constituted by a film impregnated by bacteriostatic and bactericidal agents.

3. A doormat, according to claim 1, characterized in that said block of sheet elements (7) comprises a bottom film (8) and a top protective film (9).

4. A doormat, according to claim 1, characterized in that said cleaning portion (3) is removably associated with said base (2).

5. A doormat, according to claim 1, characterized in that said cleaning portion (3) is associated with said base (2) by means of snap fasteners (6).

6. A doormat, according to claim 1, characterized in that said base (2) is made of a rubber material.

7. A doormat, according to claim 1, characterized in that said cleaning portion (3) is made of a microfiber material.

8. A doormat, according to claim 1, characterized in that said doormat comprises decorative elements.

9. A doormat, according to claim 1, characterized in that said base constituting said bristle cleaning portion forms an integrating inlet part of said doormat and said sheet element block is a sheet element block covering the whole or a portion of the outlet part of said doormat.
